(19) United States
(12) Patent Application Publication
(10) Pub. No.: US 2008/0299014 A1
(43) Pub. Date: Dec. 4, 2008

(54) AROMA DIFFUSER FOR VEHICLES

(76) Inventor: Yong Chul Kim, Hwaseong-si (KR)

Correspondence Address: MORGAN, LEWIS & BOCKIUS LLP (SF), One Market, Spear Street Tower, Suite 2800, San Francisco, CA, 94105, US

(21) Appl. No.: 11/861,531

(22) Filed: Sep. 26, 2007

(30) Foreign Application Priority Data
May 28, 2007 (KR) 10-2007-0051478

(51) Int. Cl. A61L 9/12; A61L 9/015

(52) U.S. Cl. 422/124

(57) ABSTRACT

An aroma diffuser includes a housing, made up of a base, attached to a roof panel in a passenger compartment of a vehicle; and a cover coupled to the base. The cover has an inlet port, through which air in the passenger compartment is drawn, and an outlet port, through which the air is discharged. A motor and a fan are provided in the housing, the fan being operated by the motor. A filter is provided in the housing near an inlet of the fan. An aromatic-substance-containing case is provided in the housing near the fan. An aromatic substance is stored in the case, and the case is opened when the fan operates. A pollution sensor and a control unit are also provided in the housing. The control unit receives a signal from the pollution sensor, and controls the fan and the case.

### CROSS REFERENCE TO RELATED APPLICATIONS

This application claims priority to Korean Application No. 10-2007-0051478, filed on May 28, 2007, the disclosure of which is incorporated herein by reference.

### BACKGROUND OF THE INVENTION

1. Field of the Invention

The present invention relates to an aroma diffuser for vehicles which has an aroma dispensing function and an air purifying function.

2. Description of the Related Art

Generally, aroma diffusers are placed in passenger compartments of vehicles to eliminate odor and reduce air pollution levels. Such aroma diffusers are classified as either volatilization type and or forced volatilization type.

A natural volatilization type aroma diffuser includes a casing containing an aromatic substance. The aromatic substance is naturally volatilized through a dispensing hole in the casing. The amount of aromatic substance that is volatilized is high during summer, and low during winter. Therefore, the aromatic substance is consumed quickly in the summer, and aroma is not evenly dispersed throughout the entire passenger compartment in the winter.

A forced volatilization type aroma diffuser includes an aromatic substance casing mounted to the grill of an air conditioner outlet, such that the aromatic substance is volatilized by air discharged through the outlet. Therefore, when the air conditioner is not in operation, the aromatic substance is not satisfactorily volatilized.

Further, neither type purifies air.

### SUMMARY OF THE INVENTION

An aroma diffuser includes a housing, made up of a base, attached to a roof panel in a passenger compartment of a vehicle; and a cover coupled to the base. The cover has an inlet port, through which air in the passenger compartment is drawn, and an outlet port, through which the air is discharged. A motor and a fan are provided in the housing, the fan being operated by the motor. A filter is provided in the housing near an inlet of the fan. An aromatic-substance-containing case is provided in the housing near the fan. An aromatic substance is stored in the case, and the case is opened when the fan operates. A pollution sensor and a control unit are also provided in the housing. The control unit receives a signal from the pollution sensor, and controls the fan and the case.

The case may be opened at periodic intervals for a predetermined amount of time every interval. The opening period and/or the amount of time may be controlled based on the air pollution level detected by the pollution sensor, and/or an ignition ON/OFF state of the vehicle. The speed at which the fan operates may also be controlled based on the ignition ON/OFF state of the vehicle and/or the air pollution level.

A lamp may be provided on the cover. The color emitted by the lamp may be controlled based on the speed at which the fan operates. An ion generator may also be provided in the housing near an outlet of the fan.

The aroma diffuser may be supplied with power from a main vehicle battery when the vehicle is in the ignition ON state, and supplied with power from a subsidiary battery when the vehicle is in the ignition OFF state.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a perspective view showing the external appearance of an aroma diffuser for vehicles, according to an embodiment of the present invention;

FIG. 2 is an exploded perspective view of the aroma diffuser of FIG. 1;

FIG. 3 is a perspective view of the aroma diffuser of FIG. 1; partially assembled, and

FIG. 4 is a block diagram illustrating operation of an aroma diffuser according to an embodiment of the present invention.

### DESCRIPTION OF THE PREFERRED EMBODIMENTS

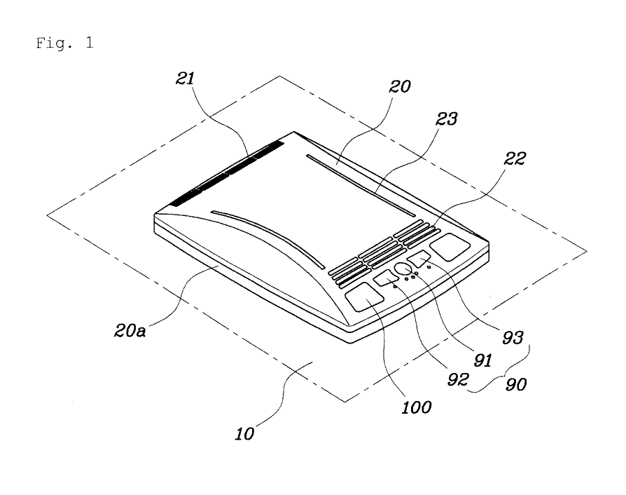

As shown in FIG. 1, an aroma diffuser according to exemplary embodiments of the present invention includes a base 20a mounted to a roof panel 10 of a vehicle, and a cover 20, which covers the base 20a and is removably coupled thereto. The base 20a and the cover 20 form the housing of the aroma diffuser. Various components are installed in the housing.

An inlet port 21 and an outlet port 22 are provided at opposite longitudinal ends of the cover 20. A room lamp 100 and various control switches 90 are provided in the cover 20 around the outlet port 22. Mood lamps 23 are provided at opposite lateral ends of the cover 20. The mood lamps 23 may change color depending on the flow rate of air. For example, the mood lamps 23 may be constructed such that, during low flow, they emit green light, and, during high flow, they emit blue light.

The base 20a is disposed in the center of the passenger compartment or between the driver's seat and the passenger seat. Therefore, the aroma diffuser can evenly supply purified air into the passenger compartment. Furthermore, because a room lamp is integrated with the aroma diffuser, space is utilized efficiently.

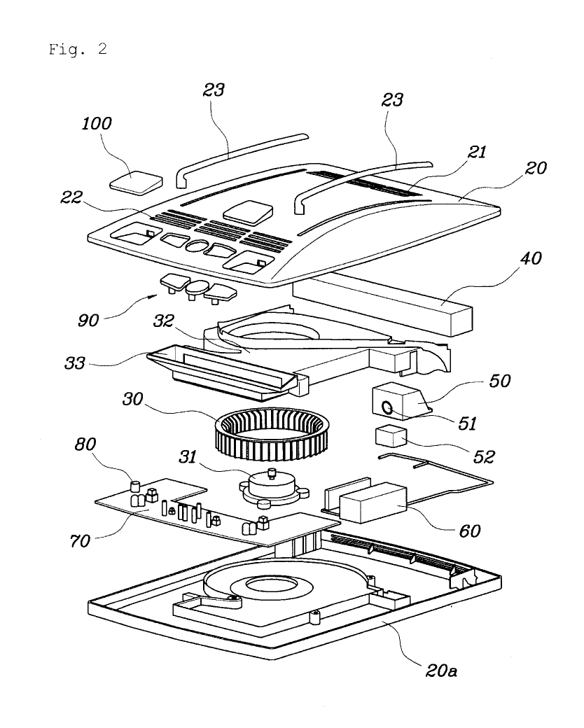

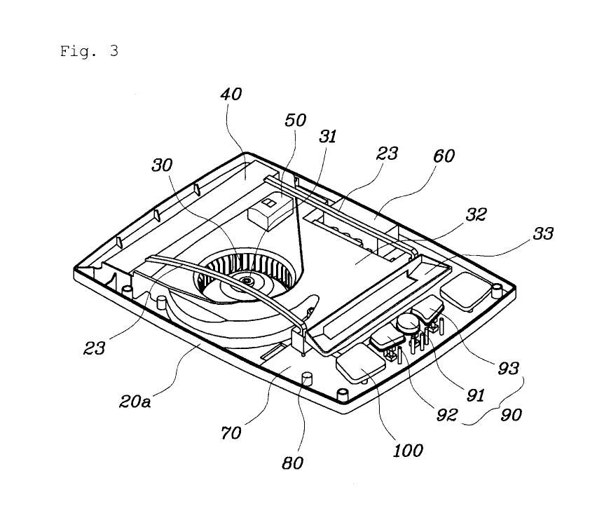

Referring to FIGS. 2-3, a fan 30, which is operated by a motor 31, is provided on the base 20a. When the fan 30 is operated, air in the passenger compartment is drawn into the aroma diffuser through the inlet port 21, and is thereafter discharged through the outlet port 22. A filter 40 is provided adjacent the inlet of the fan 30. An aromatic-substance-containing case 50 is provided adjacent the fan 30. In addition, an ion generator 60 is provided downstream of the aromatic-substance-containing case 50. Thus, air, which is drawn into the aroma diffuser, is purified by the filter 40 and, thereafter, is discharged along with aroma and ions through the discharge port 22.

The aromatic-substance-containing case 50 is airtight. A solid aromatic substance is stored in the aromatic-substance-containing case 50. It is preferable that a natural aromatic substance such as a phytoncide be used as the solid aromatic substance. A solenoid 52 opens or closes a dispensing hole 51 in the aromatic-substance-containing case 50 when the fan is on. The solenoid 52 and dispensing hole 51 can be designed and implemented by a person of ordinary skill in the art based on the teachings herein.

A circuit board 70 for controlling electric components is provided on the base 20a. The circuit board 70 comprises a control unit, which may include a processor, memory, and associated hardware, software, and/or firmware as may be selected and programmed by a person of ordinary skill in the art based on the teachings herein. Furthermore, a pollution sensor 80 is provided on the circuit board 70. When the sensor 80 detects that the level of air pollution in the passenger compartment is greater than a reference level, the control unit 70 turns on the fan 30. Simultaneously, the control unit 70 opens the aromatic-substance-containing case 50 and turns on the ion generator 60.

A support member 32 mounts the fan 30 to the base 20a. Air is discharged to the outlet port 22 through a discharge hole 33 in the support member 32.

The aroma diffuser can be automatically or manually operated. In the manual method, a user manipulates the control switches 90, such that the fan 30 is turned on and/or the aromatic-substance-containing case 50 is opened or closed. In the illustrated embodiment, referring to FIGS. 1 and 3, switches 91-93 may be any combination of a mode (automatic/manual) switch, a room lamp ON/OFF switch, an aromatic-substance-containing case opening/closing switch, and/or one or more switches for turning the fan 30 on and off, and adjusting the rotating speed thereof.

In the automatic method, when the sensor 80 detects that the level of air pollution in the passenger compartment is greater than a reference level, the aroma diffuser is turned on. Alternatively, the aroma diffuser may be turned on at periodic intervals for a predetermined amount of time every repetition. Furthermore, the operating period and/or operating time of the aroma diffuser may vary in response to the ignition ON/OFF state of the vehicle or the pollution level detected by the sensor 80. For example, in the ignition ON state of the vehicle, power is supplied to the aroma diffuser from the main vehicle battery. In the ignition OFF state, power is supplied to the aroma diffuser from a subsidiary battery. The subsidiary battery may be solar powered.

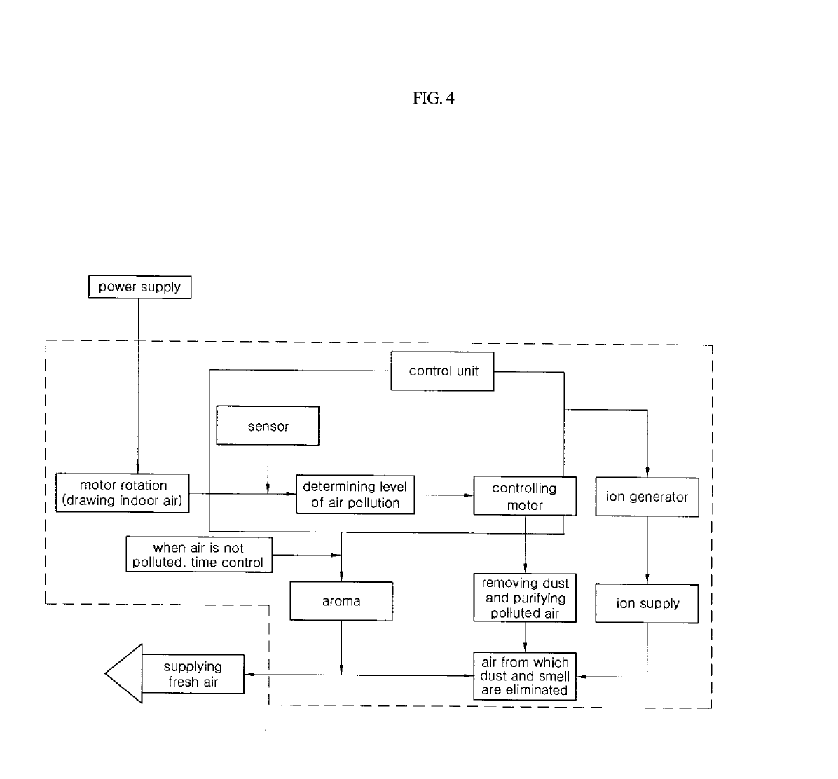

An exemplary operation of the aroma diffuser will be described herein below with reference to the above drawings and FIG. 4.

When the control unit 70 determines that the air in the passenger compartment is polluted (using the sensor 80), the aroma diffuser is turned on. If the control unit 70 determines that the air in the passenger compartment is not polluted, the aroma diffuser is operated at periodic intervals for a predetermined amount of time every repetition.

In the case where the air is polluted, the control unit 70 operates the motor 31 such that pollutant is eliminated by the filter 40. Furthermore, the control unit 70 operates the solenoid 52 to open the aromatic-substance-containing case 50, and operates the ion generator 60.

Further, when the air in the passenger compartment is polluted, the control unit 70 may control the aroma diffuser based on the pollution level; e.g., the control unit 70 controls the speed at which the fan 30 rotates, and controls the opening/closing period of the aromatic-substance-containing containing case 50 and the time for which it is opened based on the pollution level.

As described above, embodiments of the present invention provide an aroma diffuser which supplies purified air into the passenger compartment along with aroma and ions, thus providing pleasant air to a driver and passengers. Furthermore, because the aroma diffuser is provided under the ceiling of the passenger compartment, the air can be evenly supplied throughout the passenger compartment. In addition, because the aroma diffuser is operated independently from the air conditioner, it can be freely used regardless whether or not the conditioner is being operated. As well, a room lamp may be integrated with the aroma diffuser, utilizing space efficiently. Moreover, the mood lamp may provide an even more pleasant environment to the driver and passengers. Furthermore, because the ON/OFF operation of the aroma diffuser is appropriately controlled in response to its surroundings, the aromatic substance is prevented from being wasted.

Although the preferred embodiment of the present invention has been disclosed for illustrative purposes, those skilled in the art will appreciate that various modifications, additions and substitutions are possible, without departing from the scope and spirit of the invention as disclosed in the accompanying claims.

What is claimed is:

1. An aroma diffuser, comprising:
a housing, comprising:
a base configured to be attached to a roof panel in a passenger compartment of a vehicle; and
a cover coupled to the base, the cover having an inlet port, through which air in the passenger compartment is drawn, and an outlet port, through which the air is discharged;
a motor disposed in the housing;
a fan disposed in the housing, the fan being operated by the motor;
a filter disposed in the housing near an inlet of the fan;
an aromatic-substance-containing case disposed in the housing near the fan, configured to store an aromatic substance therein, wherein the aromatic-substance-containing case is opened when the fan operates;
a pollution sensor disposed in the housing; and
a control unit for receiving a signal from the pollution sensor and controlling the fan and the aromatic-substance-containing case.

2. The aroma diffuser as set forth in claim 1, wherein the aromatic-substance-containing case is opened at periodic intervals for a predetermined amount of time every interval, wherein an opening period and/or the amount of time is controlled based on an air pollution level detected by the pollution sensor and/or an ignition ON/OFF state of the vehicle.

3. The aroma diffuser as set forth in claim 1, wherein a speed at which the fan operates is controlled based on an ignition ON/OFF state of the vehicle and/or an air pollution level, detected by the pollution sensor.

4. The aroma diffuser as set forth in claim 1, further comprising a lamp on the cover for emitting at least one color.

5. The aroma diffuser as set forth in claim 4, wherein the color is controlled based on a speed at which the fan operates.

6. The aroma diffuser as set forth in claim 1, further comprising an ion generator disposed in the housing near an outlet of the fan.

7. The aroma diffuser as set forth in claim 1, wherein the aroma diffuser is supplied with power from a main vehicle battery when the vehicle is in an ignition ON state, and is supplied with power from a subsidiary battery when the vehicle is in an ignition OFF state.
